(19) United States
(12) Patent Application Publication
(10) Pub. No.: US 2011/0091904 A1
(43) Pub. Date: Apr. 21, 2011

(54) METHOD FOR DETERMINATION OF AFFINITY AND KINETIC CONSTANTS

(75) Inventors: Mats Inganas, Uppsala (SE); Josefin Bolik, Uppsala (SE); Johan Engstroem, Uppsala (SE); Karolina Osterlund, Uppsala (SE)

(73) Assignee: GYROS PATENT AB, Uppsala (SE)

(21) Appl. No.: 12/920,735

(22) PCT Filed: Mar. 5, 2009
(86) PCT No.: PCT/SE2009/050230

§ 371 Date: Jan. 3, 2011

(30) Foreign Application Priority Data
Mar. 5, 2008 (SE) 0800514-2

(51) Int. Cl. G01N 33/53; G01N 33/00

(52) U.S. Cl. 435/7.2; 436/34

(57) ABSTRACT

The invention is related to a method for quantification of a first dissociation equilibrium constant K_{d1} for a complex AB between interactants A and B relative to a second dissociation equilibrium constant K_{d2} for a complex CD between interactants C and D.

### FIELD OF THE INVENTION

The present invention relates to a method for the determination of dissociation or affinity and kinetic constants. A method is disclosed, by which the affinity constant (Kₐ) in the interaction between two interacting molecules can be determined. The method is particularly applicable for determination of the affinity constant (Kₐ) for protein interactants that can be labelled with reagents for immobilization, e.g. biotin, and detection, e.g. suitable fluorophores. A method for the determination of the association rate constant (kₐ) for the interaction between two interacting molecules is also described. Based on experimentally determined Kₐ and kₐ the dissociation rate constant (k_{d}) can be determined.

### BACKGROUND TO THE INVENTION

The recent success in the field of biotherapeutics using therapeutic monoclonal antibodies suggests that their applicability will continue to expand into areas of unmet medical needs. Currently applications are focused on diseases in inflammation and autoimmunity, oncology and infection. In order to get regulatory approval for a new therapeutic antibody a number of properties have to be studied. These relates to either efficacy parameters or side effects that may occur during treatment of patients.

Efficacy parameters are related to desired action of the therapeutic antibody. These include the target molecule and the action that an antibody recognizing this selected target molecule may exert upon binding. Minute differences in target molecule epitope specificity may be of importance for selection of antibody candidates to create the desired action (agonistic, antagonistic, blocking etc). Of prime importance is also the affinity in the interaction between the target molecule and the antibody; the higher affinity the less amount of antibody will be needed to exert the desired action. Consequently characterization of parameters reflecting the strength of interaction is important during development, manufacturing and formulation of antibody. Considering that most antibody based therapies are designed to function over long periods of time (weeks to months) there is usually time to achieve equilibrium between target molecule (TM) and drug molecule (DM). Hence procedures for determination of affinity in solution are particularly important.

Another parameter that is important is the half life of DM in circulation. This is related to immunoglobulin subclass and potentially the degree and type of glycosylation.

Parameters affecting adverse reactions may be related to glycosylation, status of aggregation, and formulation that all may affect the immunogenic properties as well as immediate adverse reactions such as complement activation and hypersensitivity reactions, changing the affinity properties during circulation in the body.

In summary, in order to become successful, therapeutic antibodies must fulfil a number of different requirements, some of which can be traced down to the inherent antigen binding properties such as affinity and kinetic constants.

The theory for describing affinity interactions is well known and accepted. Molecular interactions can be described as two reactions:

The association reaction (A+B [symbol] AB), and the dissociation reaction (AB [symbol] A+B), where

A=ligand
B=receptor
AB=complex formed by ligand and receptor

The dissociation constant (K_{d}) for the interactions between two interacting molecules is related to the concentration of the interactants as follows:

K_{d}=[A][B]/[AB] Eq. 1

where
[A]=ligand A concentration
[B]=receptor B concentration
[AB]=concentration of complex formed by ligand A and receptor B

The conservation of mass law provides that the total amount of ligand (A₀) is constant, and for the reaction provided in Eq. 1 the total amount ligand (A₀) can be expressed by the use of the following equation:

A₀=A_{f}+AB_{f} Eq. 2

where
A₀ is the total amount ligand A
A_{f} is the amount of free ligand A
AB_{f} is the amount of the complex formed by ligand A and receptor B

Correspondingly, the total amount receptor (B₀) can be expressed by the use of the following equation:

B₀=B_{f}+AB_{f} Eq. 3

where
B₀ is the total amount receptor B
B_{f} is the amount of free receptor B
AB_{f} is the amount of the complex formed by ligand A and receptor B

Accordingly, by combining Eq. 1-3, the equilibrium equation for the amount of free receptor (B) can be expressed as:

B_{f}=½((B₀−K_{d}−A₀)+√((B₀)²+2B₀K_{d}−2B₀A₀+((K_{d})²)+2A₀K_{d}+(A₀)²)) Eq. 4

Assuming linearity of the signal measured for B (Signal_{B}_{_{—}}_{measured}) can be expressed by the following equation:

Signal_{B}_{_{—}}_{measured}=(Signal_{B}_{_{—}}_{100%}−Signal_{B}_{_{—}}_{0%})B_{f}/B₀+Signal_{B}_{_{—}}_{0%}

where
Signal_{B}_{_{—}}_{measured} is the signal measured for B_{f}
Signal_{B}_{_{—}}_{100%} is the signal measured for B_{f} when all B is free
Signal_{B}_{_{—}}_{0%} is the signal measured for B_{f} when all B is bound in the complex

Combination of Eq. 4 and Eq. 5 provides the following (Eq. 6):

Signal_{B}_{_{—}}_{measured}=((Signal_{B}_{_{—}}_{100%}−Signal_{B}_{_{—}}_{0%})/2B₀)((B₀−K_{d}−A₀)+√((B₀)²+2B₀K_{d}−2B₀A₀+((K_{d})²)+2A₀K_{d}+(A₀)²))+Signal_{B}_{_{—}}_{0%}

The affinity constant (Kₐ) and the dissociation constant (K_{d}) for the interactions between two interacting molecules are related as follows:

Kₐ=1/K_{d} Eq. 7

The dissociation constant (K_{d}) for the interactions between two interacting molecules is related to the reaction rate constants for the association reaction and the dissociation reaction as follows:

K_{d}=k_{d}/kₐ Eq. 8

kₐ=reaction rate constant for the association reaction
k_{d}=reaction rate constant for the dissociation reaction

There are essentially two approaches for determining affinity related parameters (Kₐ, K_{d}, kₐ and k_{d}) for interacting biomolecules (sometimes called biomolecular interactants, or interactants).

### Approach 1—Determination of K_{d}.

In the first approach, K_{d} and the association rate constant (kₐ) are determined by use of the experiments outlined below, and then, by combining K_{d} and kₐ, k_{d} can be calculated (Eq. 8). Further, Kₐ is calculated from Eq. 7.

For determination of K_{d}, a constant amount of one interactant (for example interactant A in equations 1-6) is mixed with varying amounts of the other interactant (for example interactant B in equations 1-6) until equilibrium is reached. It may require several days to reach equilibrium, depending on affinity (the higher affinity the longer time is usually needed to reach equilibrium). After equilibrium has been reached, the amount of free B can be determined. Following this measurement of B_{f}, Eq. 6 can be used to calculate K_{d}. Typical data obtained in such an experiment are shown in FIG. 1.

There are many different options to determine the amount of free interactant, however, the determination of the free interactants should be rapid, in order to minimize the impact upon the equilibrium of the reaction.

### Approach 1—Determination of Kinetic Reaction Rate Constants.

For determination of the association rate constant (kₐ), constant amounts of both interactants are mixed, allowing complex formation to occur under time limitations (i.e. the formation of the complex is not allowed to reach equilibrium conditions). Either of the free interactants can be determined in a subsequent analysis. This procedure requires strict control of time elapsed between mixing and analysis and will generate data on association rate constant of interaction. Thus, for the association reaction and the dissociation reaction described above the following kinetic equations are given:

d[A]/dt=k_{d}*[AB]−kₐ*[A]*[B] Eq. 9a

d[B]/dt=k_{d}*[AB]−kₐ*[A]*[B] Eq. 9b

d[AB]/dt=−k_{d}*[AB]+kₐ*[A]*[B] Eq. 9c

where
[A]=ligand concentration
[B]=receptor concentration
[AB]=concentration of complex formed by ligand and receptor
d[A]/dt=the change of concentration of ligand A per time unit
kₐ=reaction rate constant for the association reaction
k_{d}=reaction rate constant for the dissociation reaction

At equilibrium conditions for the above reaction, the following applies:

d[A]/dt=d[B]/dt=d[AB]/dt=0 Eq. 10

From a series of experiments wherein the time for complex formation is varied and strictly controlled, the association rate constant (kₐ) can be determined.

At the start of the reaction, [AB]=0, thus the following boundary conditions apply::

$$\frac{d A}{d {t}_{t = 0}} = - {k}_{a} \cdot {A}_{0} \cdot {B}_{0}$$

$$\frac{d B}{d {t}_{t = 0}} = - {k}_{a} \cdot {A}_{0} \cdot {B}_{0}$$

$$\frac{d AB}{d {t}_{t = 0}} = {k}_{a} \cdot {A}_{0} \cdot {B}_{0}$$

Typical data from such an experiment are shown in FIG. 2. By combining K_{d} and kₐ, k_{d} can be calculated (Eq. 8).

Approach 2—Direct Measurement of kₐ and k_{d}.

In the second approach, one of the interactants is immobilized to a solid phase. The other interactant flows over the surface while the interaction is monitored in real time (Biacore X100 and its analogues). In this format association and dissociation rate constants (kₐ and k_{d}) are experimentally determined and the affinity equilibrium constants (Kₐ and K_{d}) are calculated (Eq. 7-8).

Today surface based procedures have been established as the primary methodology for determination of association and dissociation rate constants primarily because of availability of the Biacore system. However, when working with high affinity interactions (nM to pM) the dissociation rate constant can be very small. In the paper referred to below, the authors describe a monoclonal antibody where the dissociation rate constant is 1.1×10⁻⁵ generating a calculated K_{d} of 4.0 pM.

In order to monitor the dissociation phase in Biacore X100, the experiments have to be extended to several or even many hours to collect the data for accurate determination of the k_{d} (for example 3-4 hours, as described in A. W. Drake et al., Anal. Biochem. 328 (2004) 35-43). Another well known interaction, biotin-SA has been studied in this respect by Piran U, Riordan W J., J Immunol Methods. 1990 Oct. 4; 133(1):141-3. ^{••}The dissociation rate constant for underivatized streptavidin was 2.4×10(−6) s−1, or approximately 30-fold higher than that observed for egg avidin 7.5×10(−8) s−1). So in summary the dissociation rate constant can be in the order of 10⁻⁸ s⁻¹. This has obvious implications on system occupancy when running sequential experiments to provide a complete data set.

The possibility to perform determinations of affinity and kₐ in solution becomes particularly interesting when the affinities of drug molecule candidates regularly are in the nM to pM range (drug molecules are hereinafter referred to as DM). However, currently available equipment (KinExA from Sapidyne, as described in A. W. Drake et al., Anal. Biochem. 328 (2004) 35-43) suffers from a number of limitations: Essentially only sequential assay procedures are possible using the equipment available, which makes the analytical procedure time consuming. Additionally, the procedure consumes relatively large amounts of material. According to Drake et al, a sample volume of 5 mL was drawn through the flow cell in a K_{D}-controlled experiment, and a sample volume of 500 μl was analyzed for the antibody controlled experiment. Both these technical disadvantages also add significant cost to the overall test procedure. The relatively large sample amounts needed also makes it difficult and costly to perform replicate experiments and/or to generate more data points for improved curve fit.

### SUMMARY OF THE INVENTION

The present invention is related to a method for quantification of a first dissociation equilibrium constant K_{d1} for a complex AB relative to a second dissociation equilibrium constant K_{d2} for a complex CD, wherein the complex AB is formed by an association reaction between two interactants A and B, and wherein the complex AB can dissociate to form the interactants A and B, and wherein the complex CD is formed by an association reaction between two interactants C and D, and wherein the complex CD can dissociate to form the interactants C and D, wherein the method comprises the steps:

a) a microfluidic device comprising a plurality of microchannel structures is provided, wherein
i) at least one of the microchannel structures comprises a first capturer immobilized therein, wherein the first capturer is capable of binding to one of the interactants A or B;
ii) at least one of the microchannel structures comprises a second capturer immobilized therein, wherein the second capturer is capable of binding to one of the interactants C or D;
b) a constant amount of interactant A is mixed with varying amounts of interactant B, each mixture comprising A and B is allowed to react to form the complex AB and the mixture comprising interactant A, interactant B and the complex AB is contacted with the first capturer, so that the first capturer binds to one of the interactants A or B;
c) a constant amount of interactant C is mixed with varying amounts of interactant D, each mixture comprising C and D is allowed to react to form the complex CD and the mixture comprising interactant C, interactant D and the complex CD is contacted with the second capturer, so that the second capturer binds to one of the interactants C or D;
d) the amount of at least one of the interactants A or B, or the complex AB, is determined, and a first dataset is determined which characterises the reaction between interactant A and interactant B;
e) the amount of at least one of the interactants C or D, or the complex CD, is determined, and a second dataset is determined which characterises the reaction between interactant C and interactant D;
f) the first dataset is compared to the second dataset in order to obtain quantification of K_{d1} compared to K_{d2}.

The present invention is further related to a method for quantification of a dissociation equilibrium constant K_{d1} for a complex AB relative to a dissociation equilibrium constant K_{d2} for a complex CD, wherein the complex AB is formed by an association reaction between two interactants A and B, and wherein the complex AB can dissociate to form the interactants A and B, and wherein the complex CD is formed by an association reaction between two interactants C and D, and wherein the complex CD can dissociate to form the interactants C and D, wherein the method comprises the steps:

a) an amount of interactant A is immobilized in a first set of microchannel structures;
b) an amount of interactant C is immobilized in a second set of microchannel structures;
c) in the first set of microchannel structures varying amounts of interactant B are contacted with the immobilized interactant A and is allowed to react to form the complex AB, so that the complex AB is immobilized;
d) in the second set of microchannel structures varying amounts of interactant D are contacted with the immobilized interactant C and is allowed to react to form the complex CD, so that the complex CD is immobilized;
e) for each amount of interactant B contacted with the immobilized interactant A the amount of the immobilized complex AB is determined to obtain a first dataset characterising the interaction between the interactants A and B;
f) for each amount of interactant D contacted with the immobilized interactant C the amount of the immobilized complex CD is determined to obtain a first dataset characterising the interaction between the interactants C and D;
g) the first dataset is compared to the second dataset in order to obtain quantification of K_{d1} compared to K_{d2}.

The present invention is further related to a method for the determination of the reaction rate constant kₐ for the association reaction between two interactants A and B forming a complex AB, and for the determination of the dissociation equilibrium constant K_{d} for the complex AB dissociating to form the interactants A and B, wherein

a) the dissociation equilibrium constant K_{d} for the complex AB is determined by mixing a constant amount of interactant A with varying amounts of interactant B, each mixture comprising A and B is allowed to reach equilibrium for the reaction to form the complex AB, after equilibrium has been reached for the reaction the amount of at least one of the interactants A and B, or the complex AB, is determined by the use of a first analytical method;
b) the reaction rate constant kₐ for the association reaction between two interactants A and B forming a complex AB is determined by mixing predetermined amounts of the interactants A and B under time restricted and time controlled conditions so that the reaction to form the complex AB is not allowed to reach equilibrium conditions, after mixing the interactants A and B in a controlled time interval at least one of the interactants A and B, or the complex AB, is determined by the use of a second analytical method;
wherein the first and the second analytical method comprises the steps of
i) applying a sample volume to a chromatography column with pre-disposed chromatography particles, wherein the chromatography column has a volume less than 100 nl
ii) capturing at least one of the interactants in the chromatography column.

The present invention is further related to a method wherein the first and/or the second analytical method is a SIA method, or an IAA method, or a BIA method.

The present invention is further related to a method wherein the pre-disposed chromatography particles has an average diameter less than 100 μm, preferably less than 60 μm, more preferably less than 30 μm, and even more preferably less than 20 μm, such as 15 μm, or less than 10 μm, preferably less than 5 μm, more preferably less than 1 μm.

The present invention is further related to a method further comprising the step of removing disturbing components before performing said first and second analytical method.

The present invention is further related to a method wherein at least one of the interactants A and B comprises a molecule bound to a cell membrane.

The present invention is further related to a method wherein the chromatography column used in the first and/or second analytical method is incorporated in a microfluidic device comprising a plurality of microchannel structures.

The present invention is further related to a microfluidic device comprising a plurality of microchannel structures for use in a method according to the invention, wherein said microchannel structures comprise a chromatography column with a volume of less than 100 nl.

The present invention is further related to a microfluidic device wherein said microchannel structures further comprise a mixing chamber upstream of the chromatography column.

The present invention is further related to a micro fluidic device wherein said microchannel structures further comprise means for removing disturbing components upstream of the chromatography column.

The present invention is further related to a micro fluidic device wherein said mixing chamber has a volume less than 5 μl, preferably less than 1000 nl, more preferably less than 200 nl, even more preferably less than 20 nl, or less than 10 nl, or less than 1 nl.

### BRIEF DESCRIPTION OF THE DRAWINGS

FIG. 1. Determination of K_{d}. x-axis: Amount of B added. y-axis: Fluorescent signal measured.

FIG. 2. Determination of kₐ. x-axis: Time (s). y-axis: Measured amount of B.

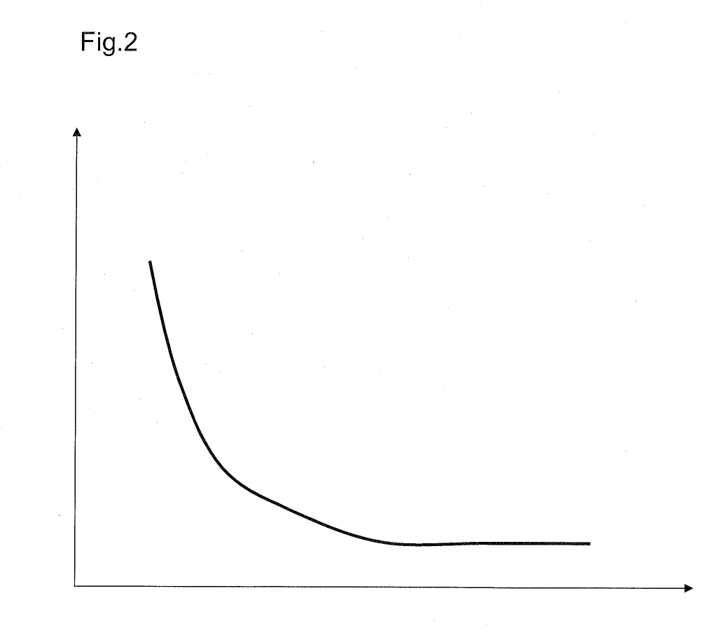

FIG. 3: Percent of free antibody vs. concentration of TSH, data points and curve fit from Example 1.

FIG. 4: Response generated from the amount of free antibody captured on the solid phase followed over time in a reaction mixture of antigen and antibody, from Example 2.

FIG. 5: Results from Example 3. x-axis: Antibody concentration (ng/ml). y-axis: Fluorescent signal measured.

### DETAILED DESCRIPTION OF THE INVENTION

Analysis of Affinity after Equilibration of Interactants has been Reached in Solution.

In one embodiment of the invention, for determination of Kₐ and K_{d}, a constant amount of one interactant (for example DM) is mixed, for example in a microtiter plate well, with varying amounts of the other interactant (for example a target molecule, hereinafter referred to as TM) until equilibrium is reached. It may require several days to reach equilibrium, depending on affinity (the higher affinity the longer time is usually needed to reach equilibrium). The amount of either of the free interactants is determined by subsequent analysis. Kₐ and K_{d} can be calculated based on the data obtained. There are many different options to determine the amount of free interactant.

In one embodiment of the invention a Gyrolab Bioaffy® CD (Gyros AB, Uppsala, Sweden) can be used to perform multiplexed parallel analysis of a multitude of samples (for example 112), where the proportions of interactants can differ between the samples. A Gyrolab Bioaffy® CD is a disc having a compact disc format, wherein the disc comprises one or more microchannel structures suitable for transport and mixing of fluids. The CD can rotate so that fluids are propagated through the microchannel structures due to the centripetal force.

Gyrolab Bioaffy® CD's utilize only minute amounts of interactants allowing significantly reduced consumption of interactants compared to alternative procedures. In alternative embodiments of the invention the sample volume can be less than 5 μl, preferably less than 1000 nl, more preferably less than 200 nl, even more preferably less than 20 nl.

The time for analysing a complete data set for determining affinity of interactions is less than 60 min in accordance with the ordinary process time for Gyrolab Bioaffy® reactions, which is a significantly shorter time than what has been published for alternative procedures.

For determination of the association rate constant (kₐ), constant amounts of both interactants are mixed, allowing complex formation to occur under time limited conditions (i.e. the formation of the complex is not allowed to reach equilibrium conditions). Either of the free interactants are determined in a subsequent analysis. This procedure requires strict control of time elapsed between mixing and analysis and will generate data on association rate constant (kₐ) of interaction.

From a series of experiments wherein the time for complex formation is varied and strictly controlled, the association rate constant (kₐ) is determined. By combining K_{d} and kₐ, k_{d} is calculated.

### Determination of the Interactants (DM and TM).

The sample containing DM and TM can be analysed for either of the free form of the two interactants by modifying the assay conditions accordingly. Thus the free form of TM is preferentially determined in a SIA assay (sandwich immunoassay) and the free form of DM is preferentially determined in IAA (indirect antibody assay). The analysis process can focus on either of these two interactants (DM or TM), or it can determine both interactants (DM and TM) in the same run using the two different assay formats (SIA and IAA). In one embodiment of the invention, the analysis is done in a vessel enabling multiplexed analysis The vessel can for example be a CD formed vessel, also called a CD, which is the vessel format used in Gyrolab Bioaffy® systems, wherein centripetal force is used to drive sample constituents, reagents, liquids etc. through channels in the vessel.

The response data that is generated in either of the two processes can be converted into concentrations of either of the interactants by incorporating appropriate reference curves in the batch run. This will allow elimination of technical artefacts that may occur in raw data and create more stable data for fitting data using appropriate algorithms. The inventors have found that the dose-response-curve is nonlinear for many assays. Sometimes it has been found that the dose-response-curve is S-shaped. In order to obtain good fit for nonlinear dose-response-curves it is necessary to use multiple data points for the calibration, and the data should preferably be collected evenly across the calibration range.

In one embodiment of the present invention, a curve fitted calibration curve is obtained by running several calibrant solutions. Due to the use of several calibrant solutions, it is preferable to use an analysis system that consumes only small amounts of sample or calibrant solution. Therefore it is preferable to use a miniaturized analysis system. In one embodiment of the invention, the Gyrolab Bioaffy® system is used for analysis of samples and calibrant solutions. Preferably the calibrant solutions are run in parallel in a multiplexed system, in order to save analysis time. The concentration of the calibrant solutions cover the concentration range of the analyte in the sample. Preferably the calibrant is a molecule identical to the analyte in the sample, but it can also be an analogue of the analyte in the sample if the analogue provides a similar dose-response curve.

Considering an affinity reaction between ligand A and receptor B, forming the complex AB, it has traditionally been held that it is sufficient to measure the amount of either A, B or AB, to estimate K_{d}, Kₐ, k_{d} and kₐ. However, there will always be some uncertainty related to the measurement of a given analyte, and such measurement uncertainty will often be related to the actual amount of the analyte in the sample. Usually, this measurement uncertainty is lowest for intermediate analyte concentration levels. Accordingly, the measurement uncertainty tends to increase for very low or very high analyte concentration levels. For the reaction A+B [symbol] AB it can therefore be beneficial to measure two or even all three analytes (A, B and AB) simultaneously to obtain data points with low measurement uncertainty throughout the whole reaction process. For practical reasons this can be difficult to achieve in a single run in a single channel. The use of sequential runs for such a process is time consuming, and it may also be difficult to achieve the same experimental conditions for sequential runs (in general, the reaction temperature has a great impact on the reaction rate). However, this is possible to do in a (multiplexed) parallel system wherein each analyte can be measured in separate channels, each channel operated simultaneously in a single system. Preferably, the system is a miniaturized system, so that uniform temperature conditions are easily obtained across the system. In such a case thermostating or other temperature controlling efforts are not necessary, thus the system can be non-thermostated. Thermostated systems can be complicated, and the addition of thermostating elements adds cost to such systems.

In the analysis of affinity reactions between DM and TM the use of a miniaturized system is preferable for a number of reasons. Frequently there is a limited amount of DM and TM available, and they tend to be expensive. Further, for applications involving biological samples like blood or tissue, the sample amount available can be extremely limited. Therefore it is beneficial to use a miniaturized analysis system that requires lower sample volume, or sample amount, than conventional systems. In the case of biological samples, like blood, such samples are often diluted prior to analysis, and therefore the analysis system used must have extremely high sensitivity. In one embodiment of the invention chromatographic particles are used to capture analytes, and the inventors have found that the chromatography particles used should have an average diameter less than 100 μm, preferably less than 60 μm, more preferably less than 30 μm, and even more preferably less than 20 μm, such as 15 μm. In further embodiments of the invention, the particles can have an average diameter less than 10 μm, preferably less than 5 μm, more preferably less than 1 μm.

The term chromatography particles is used to indicate the use of the particles in chromatography and is not restricted to particles only known for use in chromatography. While particles of essentially circular shape has been used in the experiments, also particles of other geometrical shapes can be contemplated for use in the invention.

The particles can be porous or non-porous.

While a chromatography column format has been used in the examples, it is understood that a slurry of particles also can be used. It is further understood that monolithic columns can be used.

The process is run as a reaction controlled system where diffusion distances for individual molecules once present in the column is not a limitation for reaction to occur and efficient analyte capture. It is believed for all practical purposes that more than 99% of available molecules are captured. This might be different compared to other systems which capture only a fraction of available molecules. This affects the detection limit of the analytical procedure.

The reason for this is probably that smaller particles have shorter interstitial distance, and therefore small particles capture molecules more efficiently than large particles, assuming that the capture efficiency is reaction controlled.

### Assay Formats (SIA, IAA, BIA).

Optionally a simultaneous analysis procedure of preformed complexes can be performed based on

(i) remaining free TM using SIA
(ii) remaining free DM with at least one TM binding arm of the DM free of interacting with immobilized TM using IAA
(iii) remaining free DM with both DM binding arms of the DM free to interact with immobilized TM and fluorophore labelled TM, respectively, using bridging immunoassay (BIA).

SIA is understood to mean sandwich immunoassay. IAA is understood to mean indirect antibody assay. BIA is understood to mean bridging immunoassay.

It is further understood that other types of immunoassays known in the art can be contemplated for use in the invention. Such immunoassays can be competitive or noncompetitive. Further, the immunoassays can be heterogeneous or homogeneous.

The design of different analysis formats (SIA, IAA, BIA) that are run simultaneously in a CD can be deduced e.g. from WO2007/108755, the entire content of which is hereby incorporated by reference. This procedure relies upon the convenient and efficient attachment of biotin labelled capture reagents to the streptavidin column forming the first layer of reactants in each of the three assay types.

There are different algorithms that are applicable for fitting raw data or data that has been converted into concentrations to calculate the affinity of interactants.

Practically, when the remaining fraction of free TM is determined after equilibrium has been reached the DM is used as capture reagent. This will prevent complexes already formed to be captured since the capture antibody will have the same epitope specificity as the DM in the complex. Similarly, when IAA or BIA is used to determine the fraction of free antibody binding sites the TM is immobilized to the solid phase.

In order to prevent preformed complexes to dissociate during passage of a capture column where capture of remaining free TM or DM will occur, the residence time of complexes in column should be kept to a minimum. In one embodiment of the invention solid particles of 15 microns in diameter are packed into a column volume of approximately 15 nl (100×250×600 micrometer) the calculated column residence time for the sample is <6 sec at a flow rate of 1 nl/s assuming the packed capture bed represents approximately 60% of available column volume. By increasing the flow rate the column residence time for the sample can be adjusted. In one embodiment of the invention the flow rate is adjusted by adjusting the rotational frequency of the CD.

### Analysis of Association Constants by Varying Time for Interaction of the Interactants.

In one embodiment of the invention a sample handling device, like for example the Gyrolab® Workstation (Gyros AB, Uppsala, Sweden), is used for aspirating and dispensing appropriate volumes of each interactant in a timely manner (constant amounts of interactants in all aliquots) to a CD containing functions for mixing pairs of liquid aliquots containing the interactants, within the CD. The mixing of the interactants within the CD microstructure can be initiated at different time points so that different mixing times for the interactants are tested on the same CD. In this manner different reaction times can be tested for complex formation between TM and DM, so that the kinetics of the reaction can be studied as described above.

In one embodiment of the invention a CD equipped with at least two individual inlet ports is used, preferentially containing volume defining units in between the inlets ports and a mixing chamber. The outlet of the mixing chamber is separated from the downstream portion of the microstructure by a valve strong enough to prevent transfer of mixed liquid to the downstream capture column under spinning conditions required for achieving mixing of the two liquids. Using such a CD device, the time elapsed from mixing the two interactants until the time for initiating analysis of free TM or DM can be controlled by the use of control software.

In one embodiment of the invention either of the free form of TM or DM is determined in a parallel analysis procedure in a similar fashion to the description above (SIA, IAA, BIA).

There are different algorithms that are applicable for fitting raw data or data that has been converted into concentrations to calculate the association rate constant (kₐ) for the interactants.

Once the two interactants have been mixed the reaction will continue and more and more complex will be formed. This process cannot be stopped and may, depending on how large volume is to be processed, association properties of the interactants, flow rate etc, potentially affect the overall outcome for affinity measurements, driving complex formation a bit longer in the last portion of sample compared to the first portion of sample. In order to avoid effects of this type the sample volume that is used for analysis should be kept to a minimum. In alternative embodiments of the invention the sample volume can be less than 5 μl, preferably less than 1000 nl, more preferably less than 200 nl, even more preferably less than 20 nl.

The mixing chamber on the CD should have a small volume in order to obtain the fast mixing necessary to enable measurements before equilibrium conditions apply, in order to measure the association rate constant. In one embodiment of the invention, the volume of the mixing chamber is 5 μl. In alternative embodiments of the invention the volume of the mixing chamber can be less than 5 μl, preferably less than 1000 nl, more preferably less than 200 nl, even more preferably less than 20 nl, or less than 10 nl, or less than 1 nl. For the mixing of a first volume of interactant A with a second volume of an interactant B, the volume of the mixing chamber should be less than 100 times the sum of the first and second volume.

It is understood that multiple separation modes can be used sequentially on the same CD, and in the same separation channel. The purpose of this can for example be to remove sample components that disturb the determination of the interactants. For example, it is understood that affinity chromatography can be used as a first separation step to remove high abundant proteins (e.g. albumin) from samples of blood, before the sample is further processed on the CD.

In one embodiment of the invention, the association rate constant is measured for the interaction between cell receptors and an analyte. Thus, one of the interactants is a cell receptor. It is understood that the invention can be used to measure avidity. It is understood that avidity is a term used to describe the combined strength of multiple bond interactions. Thus, avidity is the combined synergistic strength of bond affinities.

### EXAMPLES

### Example 1

### Determination of the Dissociation Equilibrium Constant (K_{d}) for Two hTSH (Human Thyroid Stimulating Hormone) Antibodies

### Assay Procedure:

A dilution series of TSH was mixed with an antibody with affinity for TSH. Two different antibodies were tested using the same mixing concentration. This mixture was allowed to mix 24 h in a microtitre plate to reach equilibrium condition. The mixture was loaded in the Gyrolab® Workstation (Gyros AB, Uppsala, Sweden) together with reagents and wash buffers. A standard Bioaffy® 200 CD (Gyros AB, Uppsala, Sweden) was used together with a method for a sandwich assay comprising the following steps:

Addition of wash buffer for reconditioning of the CD.
Biotinylated TSH is loaded on the streptavidine column.
Washing buffer is applied on the column.
The mixture of TSH and antibody is allowed to reach equilibrium and the mixture is applied on the column. Free antibody in the mixture is captured on the column.
Washing buffer is applied on the column.
Alexa® Fluor 647 labeled rat anti-mouse IgG monoclonal antibody is applied on the column in order to enable detection of the antibody captured on the column.
Washing buffer is applied on the column.
The detection facilities in a Gyrolab® Workstation is utilized for detection of the antibody captured on the column.

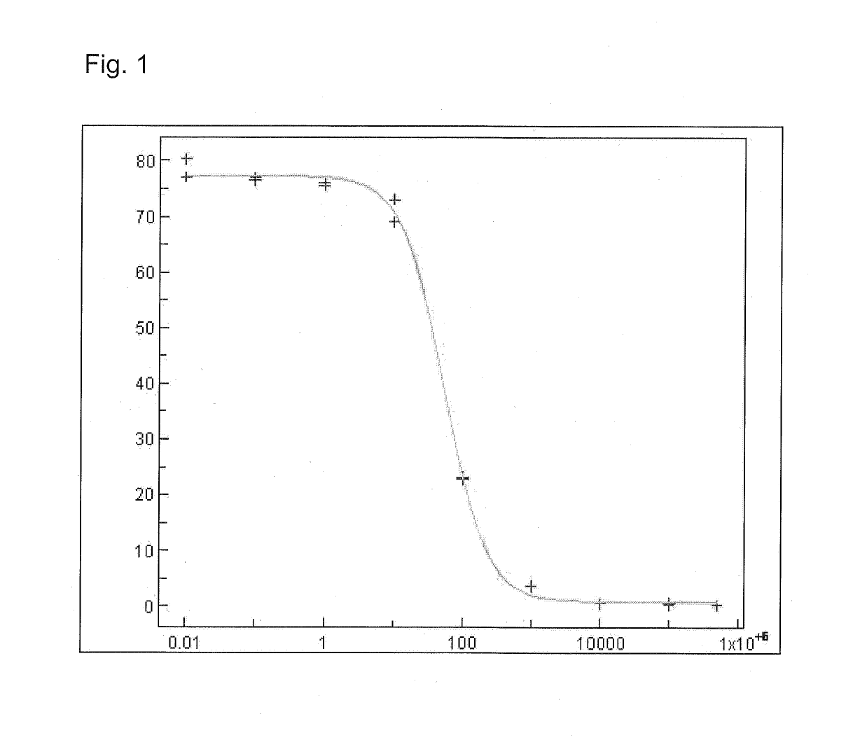

For each equilibrated mixture of TSH and antibody, three replicates were analyzed. The percent of the response from free antibody in the equilibrium mixture was plotted against the concentration of TSH, see FIG. 1. For estimating the affinity constant K_{d} the data point was fitted to a model describing the reaction.

The data points were fitted to the following equation f(x):

f(x)=((Sig100%−Sig0%)/(2*Btot))*((Btot−Kd−x)+(Btot̂2+2*Btot*Kd−2*Btot*x+Kd̂2+2*x*Kd+x̂2)̂0.5)+Sig0%

where
x is concentration of TSH.
Btot is the concentration of antibody.
Sig100% is measured signal when without TSH in the mixture
Sig0% is the measured signal when there is no free antibody.

### Reagents:

Human TSH, hTSH, (Immunometrics (UK) Ltd, London, UK)
Mab anti human TSH (clone 5401, 5404 and 5407, Medix Biochemica, Joensuu, Finland)

### EZ-Link Sulpho-NHS-LC-Biotin, Cat no 21335 (Pierce, Rockford, Ill.)

### AffiPure Goat Anti-mouse IgG, (Jacksson Immunoresearch Laboratory Inc., West Grove, Pa.)

Alexa® Fluor 647 (A-20186, Invitrogen, Täby, Sweden)

### EZ-Link Sulpho-NHS-LC-Biotin, Cat no 21335 (Pierce, Rockford, Ill.)

### Reagent Concentrations:

Biotinylated TSH conc. 100 microgram/ml
Anti TSH antibody conc. 256 pM or 512 pM binding sites for TSH
hTSH conc. 0, 4, 8, 16, 32, 128, 256, 1024, 2048, 16384, 65536, 262144 pM
Alexa labeled Goat anti mouse IgG, conc. 25 nM

### Result:

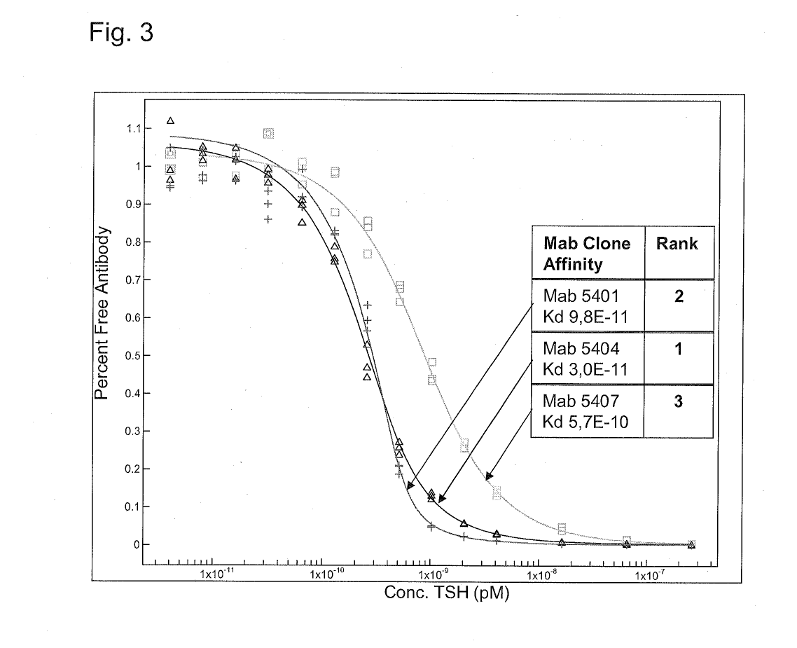

The affinity for three antibodies for TSH was determined by fitting the experimental data points, see FIG. 3, to the equation following equation f(x) described above:

For the three antibodies the following result was obtained:

TABLE 1

| | | | 95% | 95% | |
|---|---|---|---|---|---|
| | | | Confidence | Confidence | Linear. |
| Mab | K_{d} | | Limits | Limits | correlation |
| Clone | value | Std Err | Upper limit | Lower limit | coefficient |
| 5401 | 9.8E−11 | 1.3E−12 | 1.0E−10 | 9.5E−11 | 0.996 |
| 5404 | 3.0E−11 | 1.2E−12 | 3.3E−11 | 2.7E−11 | 0.991 |
| 5407 | 5.7E−10 | 2.2E−11 | 6.2E−10 | 5.3E−10 | 0.995 |

### Example 2

### Determination of Kinetic Association Constant (kₐ)

### Assay Procedure:

One concentration of TSH was mixed with one concentration of one antibody with affinity for TSH. This mixture was allowed to react during different time periods in a microtitre plate. The mixture was loaded in the Gyrolab® Workstation together with reagents and wash buffers. A standard Bioaffy® 200 CD was used together with the standard method for a sandwich assay including the following steps:

Addition of wash buffer for reconditioning of the CD.
Biotinylated TSH is loaded on the streptavidine column.
Washing buffer is applied on the column.
A mixture of TSH and antibody is applied on the column and free antibody in the mixture is captured.
Washing buffer is applied on the column.
Alexa® labeled goat anti-mouse IgG is applied on the column in order to enable detection of the antibody captured on the column.
Washing buffer is applied on the column.
The detection facilities in a Gyrolab® Workstation is utilized for detection of the antibody captured on the column.

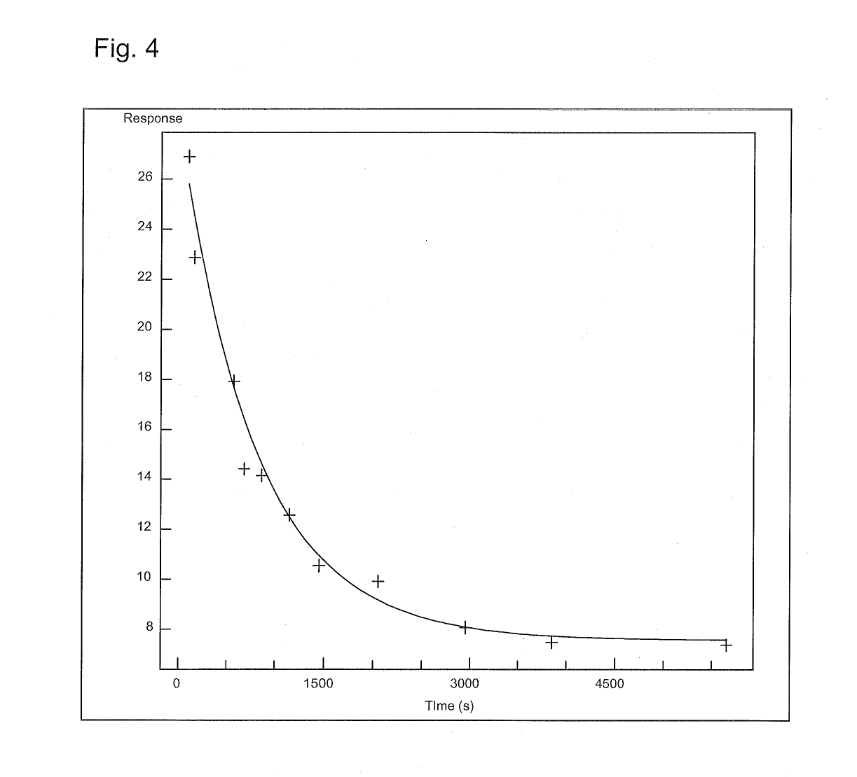

For each reaction mixture three replicates were analyzed. The response from free antibody in the mixture was plotted against the reaction time, see FIG. 4. An average for three replicates for each incubation time was fitted to a simplified model in order to extract the kinetic constant kₐ. This simplified model assumes no influence of the dissociation of the formed TSH-Antibody complex. Another assumption is that TSH is in excess, so that the concentration of TSH is not significantly changing over time. Using a more complex model without these limitations a more accurate constant may be obtained.

The data points was fitted to the following equation f(t)

f(t)=C+A*exp(k*t)=C+A*exp(kₐ*[TSHtot]*t)

where
k is the observed kinetic decay constant which is the product of the kinetic association constant, kₐ, and the starting concentration of the ligand TSH, [TSHₜₒₜ].

### Reagent Concentrations:

Biotinylated TSH, conc. 100 microgram/ml
Anti TSH antibody, conc. 256 pM or 512 pM binding sites for TSH
hTSH, conc. 2048 pM
Alexa® labeled Goat anti mouse IgG, conc. 25 nM

### Results:

The kinetic association constant (kₐ) for the TSH antibody 5407 was determined by fitting the experimental data points, see FIG. 4, to the following equation f(t) described above:

The following result was obtained:

k=0.0012 s⁻¹

kₐ=5.86*10⁶ M⁻¹s⁻¹

The linear correlation coefficient, r² is 0.966.

### Example 3

### Binding Assay on Solid Phase

### Assay Procedure

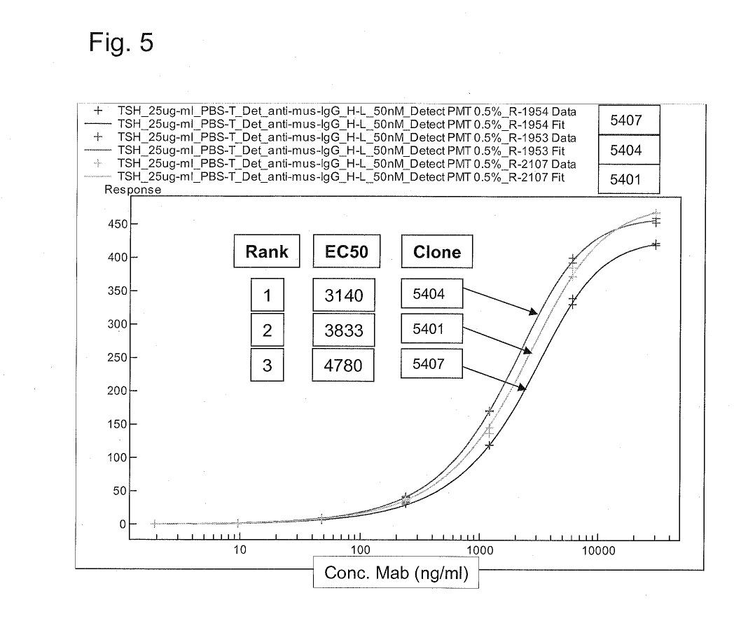

Limiting amounts of biotinylated target antigen are attached to capture columns comprising solid polystyrene (98% polystyrene and 2% divinylbenzene) particles of 15 μm diameter, wherein the particles are functionalized with streptavidin on the particle surface, followed by addition of varying amounts of antibody directed against the target antigen. Thus, a number of experiments are carried out, wherein a different amount of antibody is added in each experiment. After washing procedures the amount of bound antibody directed against target antigen is contacted with an excess of detectable Alexa® labeled rat anti-mouse IgG monoclonal. After additional washing procedures detection of the antibody captured on the column (via the labeled anti-mouse IgG monoclonal) is carried out using the detection facilities of a Gyrolab® Workstation. See FIG. 5 for results.

### Reagents

Human TSH (Immunometrics (UK) Ltd, London, UK) was biotinylated using EZ-Link Sulpho-NHS-LC-Biotin, Cat no 21335 (Pierce, Rockford, Ill.) according to standard procedures. Biotinylated TSH was diluted to 25 μg/ml in PBS-Tween and attached to 16 capture columns in the Bioaffy® 200 CD. Three mouse monoclonal antibodies directed against human TSH (clone 5401, 5404 and 5407, Medix Biochemica, Joensuu, Finland) were added at concentrations varying from 0.3 to 5000 ng/ml in standard diluent followed by detection using rat anti-mouse IgG (heavy chain-specific, clone no 3H2296, US Biological, Swampscott, Mass.) labeled with Alexa® Fluor 647 (A-20186, Invitrogen, Täby, Sweden)

### Result

The result shows affinity properties for three different antibodies, se FIG. 5. The experiment does not determine the affinity constants of the reactants but the relative position of the curves gives information regarding antibody affinity. A curve for an antibody with higher affinity (i.e. lower value of K_{d}) is shifted to the left, se FIG. 5. While a graphical representation of the reaction studied is practical for visualization purposes, it is understood that a mathematical representation of the curve can also be used in order to compare the affinities of different antibodies. For example, using curve fitting, the EC50 value can be determined for the three binders. A low EC50 value corresponds to higher affinity (i.e. lower value of K_{d}).

Definition of EC50: The term EC50 (half maximal effective concentration) refers to the concentration of a reactant (for example a drug or antibody) which induces a response halfway between the baseline signal and the maximum signal.

While the EC50 value is suitable for comparing the affinities of different antibodies, it is understood that any point of the fitted curve, positioned between the baseline signal and the maximum signal can in principal be utilized for the same purpose. It is also understood that any data point positioned in the region between the baseline signal and the maximum signal can in principal be utilized for the same purpose.

If more curves of the same type are measured using different concentrations of ligand on the solid phase kinetic and affinity properties can be calculated.

### Example 4

### Comparing Affinity Properties Obtained with Different Methods

In example 1 the affinity constants were determined in solution for three different antibodies. In example 3 affinity ranking was performed with the same three antibodies with a solid phase binding method. The affinity constants (K_{d}-values) from example 1 and the EC50 values from example 3 are listed in table 2. The affinity ranking of the three antibodies for the two methods correlates.

TABLE 2
Affinity properties for three different antibodies.

| | |
|---|---|
| Example 1 | Example 3 |

| Mab | K_{d} | K_{d} | EC50 | EC50 |
|---|---|---|---|---|
| clone | M | Rank | ng/ml | Rank |
| 5401 | 9.8E−11 | 2 | 3833 | 2 |
| 5404 | 3.0E−11 | 1 | 3140 | 1 |
| 5407 | 5.7E−10 | 3 | 4780 | 3 |

1. A method for quantification of a first dissociation equilibrium constant Kd1 for a complex AB relative to a second dissociation equilibrium constant Kd2 for a complex CD, wherein the complex AB is formed by an association reaction between two interactants A and B, and wherein the complex AB can dissociate to form the interactants A and B, and wherein the complex CD is formed by an association reaction between two interactants C and D, and wherein the complex CD can dissociate to form the interactants C and D, wherein the method comprises the steps:
a) a micro fluidic device comprising a plurality of microchannel structures is provided, wherein
i) at least one of the microchannel structures comprises a first capturer immobilized therein, wherein the first capturer is capable of binding to one of the interactants A or B;
ii) at least one of the microchannel structures comprises a second capturer immobilized therein, wherein the second capturer is capable of binding to one of the interactants C or D;
b) a constant amount of interactant A is mixed with varying amounts of interactant B, each mixture comprising A and B is allowed to react to form the complex AB and the mixture comprising interactant A, interactant B and the complex AB is contacted with the first capturer, so that the first capturer binds to one of the interactants A or B;
c) a constant amount of interactant C is mixed with varying amounts of interactant D, each mixture comprising C and D is allowed to react to form the complex CD and the mixture comprising interactant C, interactant D and the complex CD is contacted with the second capturer, so that the second capturer binds to one of the interactants C or D;
d) the amount of at least one of the interactants A or B, or the complex AB, is determined, and a first dataset is determined which characterises the reaction between interactant A and interactant B;
e) the amount of at least one of the interactants C or D, or the complex CD, is determined, and a second dataset is determined which characterises the reaction between interactant C and interactant D;
f) the first dataset is compared to the second dataset in order to obtain quantification of Kd1 compared to Kd2.

2. A method for quantification of a dissociation equilibrium constant Kd1 for a complex AB relative to a dissociation equilibrium constant Kd2 for a complex CD, wherein the complex AB is formed by an association reaction between two interactants A and B, and wherein the complex AB can dissociate to form the interactants A and B, and wherein the complex CD is formed by an association reaction between two interactants C and D, and wherein the complex CD can dissociate to form the interactants C and D, wherein the method comprises the steps:
a) an amount of interactant A is immobilized in a first set of microchannel structures;
b) an amount of interactant C is immobilized in a second set of microchannel structures;
c) in the first set of microchannel structures varying amounts of interactant B are contacted with the immobilized interactant A and is allowed to react to form the complex AB, so that the complex AB is immobilized;
d) in the second set of microchannel structures varying amounts of interactant D are contacted with the immobilized interactant C and is allowed to react to form the complex CD, so that the complex CD is immobilized;
e) for each amount of interactant B contacted with the immobilized interactant A the amount of the immobilized complex AB is determined to obtain a first dataset characterising the interaction between the interactants A and B;
f) for each amount of interactant D contacted with the immobilized interactant C the amount of the immobilized complex CD is determined to obtain a first dataset characterising the interaction between the interactants C and D;
g) the first dataset is compared to the second dataset in order to obtain quantification of Kd1 compared to Kd2.

3. A method according to claim 2, wherein the first and second sets of microchannel structures are provided in a micro fluidic device comprising a plurality of microchannel structures.

4. A method according to claim 2, wherein the amount of immobilized interactant A is the same in all the microchannel structures used.

5. A method according to claim 1, wherein interactant A is the same as interactant C.

6. A method according to claim 1, wherein the first and second capturers are immobilized in capture columns provided in the microchannel structures.

7. A method according to claim 6, wherein the capture columns comprise chromatography particles.

8. A method according to claim 7, wherein the chromatography particles are pre-disposed in the microchannel structures.

9. A method according to claim 7, wherein the chromatography particles have an average diameter less than 100 μm, preferably less than 60 μm, more preferably less than 30 μm, and even more preferably less than 20 μm, such as 15 μm, or less than 10 μm, preferably less than 5 μm, more preferably less than 1 μm.

10. A method according to claim 1, wherein at least one of the interactants A or B and/or of the interactants C or D comprises a molecule bound to a cell membrane.

11. A method according to claim 1, wherein the determination of the amount of interactant or the amount of complex is carried out by the use of a SIA method, or an IAA method, or a BIA method.

12. A method according to claim 11, further comprising the step of removing disturbing components before the determination of the amount of interactant or the amount of complex is carried out.

13. A method according to any claim 1, wherein the microfluidic device is non-thermostated.

14. The method of claim 3, wherein the amount of immobilized interactant A is the same in all the microchannel structures used.

15. The method of claim 2, wherein interactant A is the same as interactant C.

16. The method of claim 8, wherein the chromatography particles have an average diameter less than 100 μm, preferably less than 60 μm, more preferably less than 30 μm, and even more preferably less than 20 μm, such as 15 μm, or less than 10 μm, preferably less than 5 μm, more preferably less than 1 μm.

17. The method of claim 11, wherein at least one of the interactants A or B and/or of the interactants C or D comprises a molecule bound to a cell membrane.

18. The method of claim 3, wherein the microfluidic device is non-thermostated.

19. The method of claim 2, wherein the determination of the amount of interactant or the amount of complex is carried out by the use of a SIA method, or an IAA method, or a BIA method
